(19) United States
(12) Patent Application Publication
(10) Pub. No.: US 2016/0129070 A1
(43) Pub. Date: May 12, 2016

(54) Glutathione Disulfide Compositions and Related Methods for the Treatment of Cancer

(71) Applicant: South Dakota Board of Regents, Pierre, SD (US)

(72) Inventors: Xiangming Guan, Brookings, SD (US); Chandradhar Dwivedi, Brookings, SD (US); Teresa Seefeldt, Brookings, SD (US); Satya Sadhu, Telangana (IN)

(21) Appl. No.: 14/875,862

(22) Filed: Oct. 6, 2015

Related U.S. Application Data
(60) Provisional application No. 62/077,470, filed on Nov. 10, 2014.

(51) Int. Cl. A61K 38/06; A61K 45/06; A61K 9/127

(52) U.S. Cl. CPC A61K 38/063; A61K 9/127; A61K 45/06

(57) ABSTRACT

Compositions and methods for the treatment of cancer in a subject in need thereof comprising administering to the subject an effective amount of a composition comprising a GSSG and a carrier thereof. In an aspect, the carrier is a liposome. In a further aspect, the liposome is a positively charged liposome.

### CROSS-REFERENCE TO RELATED APPLICATION(S)

This application claims the benefit of U.S. Provisional Application No. 62/077,470, filed on Nov. 10, 2014; which is incorporated herein by reference in its entirety

### STATEMENT REGARDING FEDERALLY SPONSORED RESEARCH

This invention was made with government support under grant number 1R15GM093678-01 from the National Institutes of Health. The United States government has certain rights in the invention.

### FIELD OF THE INVENTION

Disclosed herein are methods and compositions for treating cancer.

### BACKGROUND OF THE INVENTION

Cancer metastasis—involving cancer cell detachment, migration, invasion, and adhesion at a site different from the original tumor—is the major cause of cancer mortality. Despite extensive research efforts, effective treatment for cancer metastasis is still lacking. Cancer cell detachment is the first and required step for metastasis. Extensive efforts have been made to develop effective treatments for metastasis by targeting various steps involved in metastasis. Compared with chemotherapy and radiation therapy, treatments derived from targeting metastatic steps have the advantage of being more selective against metastatic cells. Clinical treatments derived from targeting metastatic steps include angiogenesis inhibitors, growth factor pathway blockers, and matrix metalloproteinases (MMP) inhibitors. Additional approaches targeting metastatic steps in development include integrin inhibitors, FAK inhibitors, chemokine inhibitors that inhibit cell migration, TGF-α inhibitors, bisphosphonates and others. Nevertheless, there is a need in the art for a novel and effective treatment for metastatic cancer.

### BRIEF SUMMARY OF THE INVENTION

Disclosed herein is an anticancer composition comprising glutathione disulfide (GSSG) and a carrier thereof. In further aspects, the carrier is a liposome. In still further aspects, the liposome is a positively charged liposome.

Also disclosed is anticancer composition comprising a compound having the structure:

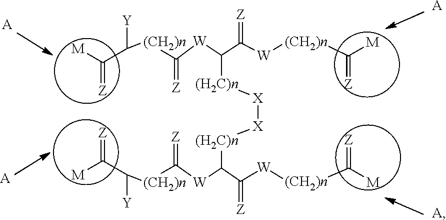

wherein A is a functional group selected from carboxylic acid, sulfonic acid, phosphoric acid, and derivatives thereof;
wherein M is selected from: —OH, —NH2, —SH, —OR, —NHR, —NR1R2, —SR, —R.;
wherein R, R1, and R2 are alkyl, alkenyl, alkynyl, cycloalkyl, cycloalkenyl, cycloalkynyl or aryl;
wherein Z is selected from O, S, and NH;
wherein Y is selected from: NH2, OH, SH, alkyl, alkenyl, alkynyl, cycloalkyl, cycloalkenyl, cycloalkynyl, and aryl;
wherein W is selected from NH, CH2, S, and O;
wherein X is selected from O, S, CH2, and NH;
wherein n is 0, 1, 2, or 3; and
a pharmaceutically acceptable carrier thereof.

In certain aspects, A is an ester or amide derivate of carboxylic acid, sulfonic acid, or phosphoric acid.

Also disclosed is a method for treating cancer in a subject, the method comprising administering to the subject an effective amount of a composition comprising GSSG or derivatives thereof and a pharmaceutically acceptable carrier thereof.

In further aspects, administration of the composition to the subject inhibits tumor cell migration. In even further aspects, administration of the composition to the subject inhibits tumor cell invasion. In still further aspects, administration of the composition inhibits tumor growth. In yet further aspects, administration of the composition induces tumor cell apoptosis.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE FIGURES

FIG. 1 shows a time course of GSSG delivered into cells by GSSG liposomes into cells.

FIG. 2A and FIG. 2B show exemplary images from cell detachment assays from NCI-H226 cells and B16-F10 cells, respectively.

FIG. 3A and FIG. 3B show exemplary images from wound healing assays from NCI-H226 cells and B16-F10 cells, respectively.

FIGS. 4A-E shows data showing exemplary effects of GSSG liposomes on the invasion property of human cancer cell lines (NCI-H226, PC-3, HT 116, and OVCAR-3) and B16-F10 murine melanoma cell line.

FIG. 5 shows exemplary images showing the effects of GSSG liposomes on tumor metastasis in lung tissue.

FIG. 6 shows tumor growth data, according to certain embodiments.

FIG. 7A and FIG. 7B show exemplary images from cell viability experiments in NCI-H226 cells and B16-F10 cells, respectively.

FIG. 8 shows data regarding the effect of GSSG liposomes on microtubule polymerization, according to exemplary embodiments.

FIGS. 9A and 9B show cell count data in response to treatment, in B16-F10 cells and NCI-H226 cells, respectively.

FIGS. 10A and 10B show exemplary images from TUNEL assays in B16-F10 cells and NCI-H226 cells, respectively.

FIG. 11 shows histograms representing cell cycle distribution in response to treatment conditions, according to certain embodiments.

FIG. 12 shows cell cycle data in response to treatment conditions according to certain embodiments.

### DETAILED DESCRIPTION

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH2CH2O— units in the polyester, regardless of whether ethylene glycol was used to prepare the polyester. Similarly, a sebacic acid residue in a polyester refers to one or more —CO(CH2)8CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A1,” “A2,” “A3,” and “A4” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dode cyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “polyalkylene group” as used herein is a group having two or more CH2 groups linked to one another. The polyalkylene group can be represented by the formula —(CH2)a-, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA1 where A1 is alkyl or cycloalkyl as defined above. “Alkoxy” also includes polymers of alkoxy groups as just described; that is, an alkoxy can be a polyether such as —OA1-OA2 or —OA1-(OA2)a-OA3, where “a” is an integer of from 1 to 200 and A1, A2, and A3 are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A1A2)C═C(A3A4) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA1A2, where A1 and A2 can be, independently, hydrogen or alkyl, cycloalkyl, alkenyl, cycloalkenyl, alkynyl, cycloalkynyl, aryl, or heteroaryl group as described herein.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)2 where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A1 or —C(O)OA1, where A1 can be alkyl, cycloalkyl, alkenyl, cycloalkenyl, alkynyl, cycloalkynyl, aryl, or heteroaryl group as described herein. The term “polyester” as used herein is represented by the formula -(A1O(O)C-A2-C(O)O)a- or -(A1O(O)C-A2-OC(O))a-, where A1 and A2 can be, independently, an alkyl, cycloalkyl, alkenyl, cycloalkenyl, alkynyl, cycloalkynyl, aryl, or heteroaryl group described herein and “a” is an interger from 1 to 500. “Polyester” is as the term used to describe a group that is produced by the reaction between a compound having at least two carboxylic acid groups with a compound having at least two hydroxyl groups.

The term “ether” as used herein is represented by the formula A1OA2, where A1 and A2 can be, independently, an alkyl, cycloalkyl, alkenyl, cycloalkenyl, alkynyl, cycloalkynyl, aryl, or heteroaryl group described herein. The term “polyether” as used herein is represented by the formula -(A1O-A2O)a-, where A1 and A2 can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The term “halide” as used herein refers to the halogens fluorine, chlorine, bromine, and iodine.

The term “hydroxyl” as used herein is represented by the formula —OH.

The term “ketone” as used herein is represented by the formula A1C(O)A2, where A1 and A2 can be, independently, an alkyl, cycloalkyl, alkenyl, cycloalkenyl, alkynyl, cycloalkynyl, aryl, or heteroaryl group as described herein.

The term “azide” as used herein is represented by the formula —N3.

The term “nitro” as used herein is represented by the formula —NO2.

The term “nitrile” as used herein is represented by the formula —CN.

The term “silyl” as used herein is represented by the formula —SiA1A2A3, where A1, A2, and A3 can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A1, —S(O)2A1, —OS(O)2A1, or —OS(O)2OA1, where A1 can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)2A1, where A1 can be hydrogen or an alkyl, cycloalkyl, alkenyl, cycloalkenyl, alkynyl, cycloalkynyl, aryl, or heteroaryl group as described herein. The term “sulfone” as used herein is represented by the formula A1S(O)2A2, where A1 and A2 can be, independently, an alkyl, cycloalkyl, alkenyl, cycloalkenyl, alkynyl, cycloalkynyl, aryl, or heteroaryl group as described herein. The term “sulfoxide” as used herein is represented by the formula A1S(O)A2, where A1 and A2 can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R1,” “R2,” “R3,” “Rn,” where n is an integer, as used herein can, independently, possess one or more of the groups listed above. For example, if R1 is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

As used herein, “Glutathione Disulfide” also referred to as “GSSG,” is the oxidized form of glutathione, having the structure:

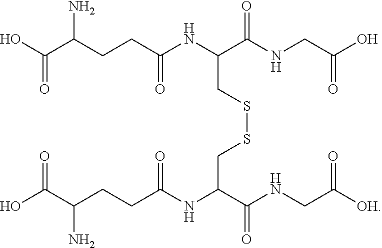 [SMILES: NC(CCC(=O)NC(CSSCC(NC(=O)CCC(N)C(=O)O)C(=O)NCC(=O)O)C(=O)NCC(=O)O)C(=O)O]

As used herein, the term “pharmaceutically acceptable carrier” or “carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, cancer of the small intestine, and cancer of the esophagus. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. In some embodiments, the present methods can be used to treat a subject having an epithelial cancer, e.g., a solid tumor of epithelial origin, e.g., lung, breast, ovarian, prostate, renal, pancreatic, or colon cancer.

As used herein, the term “subject” refers to the target of administration, e.g., an animal. Thus the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more cancer disorders prior to the administering step.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with cancer” means having been subjected to a physical examination by a person of skill, for example, a physician, and found to have a condition that can be diagnosed or treated by a compound or composition that can reduce tumor size or slow rate of tumor growth. A subject having cancer, tumor, or at least one cancer or tumor cell, may be identified using methods known in the art. For example, the anatomical position, gross size, and/or cellular composition of cancer cells or a tumor may be determined using contrast-enhanced MRI or CT. Additional methods for identifying cancer cells can include, but are not limited to, ultrasound, bone scan, surgical biopsy, and biological markers (e.g., serum protein levels and gene expression profiles). An imaging solution comprising a cell-sensitizing composition of the present invention may be used in combination with MRI or CT, for example, to identify cancer cells.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, there phrase “anti-metastatic” includes agents that prevent tumor metastasis.

The phrase “anti-cancer composition” can include compositions that exert antineoplastic, chemotherapeutic, antiviral, antimitotic, antitumorgenic, and/or immunotherapeutic effects, e.g., prevent the development, maturation, or spread of neoplastic cells, directly on the tumor cell, e.g., by cytostatic or cytocidal effects, and not indirectly through mechanisms such as biological response modification. There are large numbers of anti-proliferative agents available in commercial use, in clinical evaluation and in pre-clinical development, which could be included in this application by combination drug chemotherapy. For convenience of discussion, anti-proliferative agents are classified into the following classes, subtypes and species: ACE inhibitors, alkylating agents, angiogenesis inhibitors, angiostatin, anthracyclines/DNA intercalators, anti-cancer antibiotics or antibiotic-type agents, antimetabolites, antimetastatic compounds, asparaginases, bisphosphonates, cGMP phosphodiesterase inhibitors, calcium carbonate, cyclooxygenase-2 inhibitors, DHA derivatives, DNA topoisomerase, endostatin, epipodophylotoxins, genistein, hormonal anticancer agents, hydrophilic bile acids (URSO), immunomodulators or immunological agents, integrin antagonists, interferon antagonists or agents, MMP inhibitors, miscellaneous antineoplastic agents, monoclonal antibodies, nitrosoureas, NSAIDs, ornithine decarboxylase inhibitors, pBATTs, radio/chemo sensitizers/protectors, retinoids, selective inhibitors of proliferation and migration of endothelial cells, selenium, stromelysin inhibitors, taxanes, vaccines, and vinca alkaloids.

The major categories that some anti-proliferative agents fall into include antimetabolite agents, alkylating agents, antibiotic-type agents, hormonal anticancer agents, immunological agents, interferon-type agents, and a category of miscellaneous antineoplastic agents. Some anti-proliferative agents operate through multiple or unknown mechanisms and can thus be classified into more than one category.

According to certain aspects, disclosed is an anticancer composition comprising GSSG and a carrier thereof. Glutathione disulfide (GSSG) is an endogenous compound. It is the oxidized form of glutathione (GSH). GSH is the major endogenous antioxidant and present in mM concentration in the biological system. Structurally, GSH is a three amino acid peptide consisted of Glu-Cys-Gly (Scheme 1). The thiol or sulfhydryl group (—SH) of the cysteine residue in GSH is the key for the function of GSH. One of the functions of GSH is to protect the biological system from oxidizing species, such as reactive oxygen species (ROS). GSH achieves this by using the thiol group to reduce oxidizing species. The thiol itself is then oxidized to a disulfide (—S—S—) resulting in formation of GSSG (Scheme 1). Under the normal physiological condition, GSSG will be quickly reduced back to GSH by glutathione reductase (GR) to maintain a high ratio of GSH:GSSG in the living system. The ratio of GSH:GSSG in the living system is normally maintained at >100:1.1 Although GSSG is an endogenous molecule, it is not cell membrane permeable. The study of the impact of GSSG on cellular function and dysfunction has been hampered by a lack of an effective method to deliver GSSG into cells.

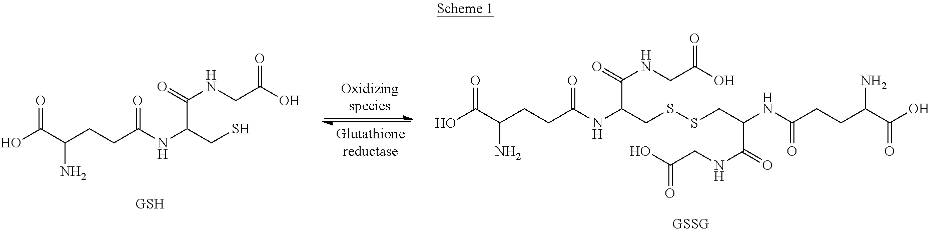 [SMILES: NC(CCC(=O)NC(CS)C(=O)NCC(=O)O)C(=O)O ; NC(CCC(=O)NC(CSSCC(NC(=O)CCC(N)C(=O)O)C(=O)NCC(=O)O)C(=O)NCC(=O)O)C(=O)O]

According to certain aspects, disclosed is an anticancer composition comprising glutathione disulfide (GSSG) and a carrier thereof. In further aspects, the carrier is a liposome. In still further aspects, the liposome is a positively charged liposome.

According to certain alternative embodiments, the carrier is a micelle. In further aspects, the micelle is comprised of a polymer or a surfactant. Nanoparticles or microparticles made of synthetic polymers, natural polymers, inorganic nanoparticles. In certain aspects, disclosed carriers are micelles. Instantly disclosed micelles may be synthetic or natural. According to certain embodiments, micelles may be polymer or surfactant based micelles.

In certain aspects, the carrier is a lipid based delivery system such as solid lipid nanoparticles, nanostructured lipids or liposomes. In certain aspects, disclosed liposome carriers may be charged or uncharged. By way of example, in certain aspects carrier liposomes are cationic, anionic or neutral. In certain embodiments, carrier liposomes are PEGylated liposomes. In certain of these embodiments, carrier liposomes are comprised at least in part of pegylated lipids.

In further embodiments, disclosed carriers are microemulsions or nanoemulsions.

In certain aspects, carriers are connected with one or more tumor targeting ligand. By way of example, in certain embodiments, tumor targeting antibodies are used to target the carrier to tumor cells.

According to certain embodiments, GSSG or derivatives thereof can be loaded into the carrier according to various methods known in the art. For example, according to certain embodiments, GSSG or derivatives thereof are complexed, conjugated or encapsulated in the carrier. In further embodiments, GSSG or derivatives thereof are conjugated or complexed to synthetic or natural polymers, including but not limited to albumin, PEG, dendrimers, and carbon nanotubes.

In further aspects, the carrier is comprised of combination systems such as polymer-lipid nanoparticles, inorganic-lipid nanoparticles or combinations of other systems.

In yet further aspects, the GSSG is complexed with the carrier. In still further aspects, the GSSG is encapsulated within the carrier. In even further aspects, GSSG is conjugated to the carrier.

In certain aspects, disclosed is an anticancer composition comprising a compound having the structure:

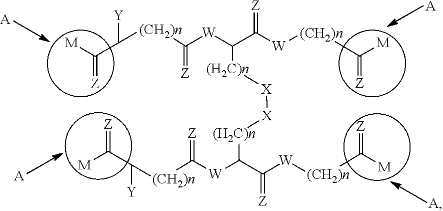

wherein A is a functional group selected from carboxylic acid, sulfonic acid, phosphoric acid, and derivatives thereof;
wherein M is selected from: —OH, —NH2, —SH, —OR, —NHR, —NR1R2, —SR, —R.;
wherein R, R1, and R2 are alkyl, alkenyl, alkynyl, cycloalkyl, cycloalkenyl, cycloalkynyl or aryl;
wherein Z is selected from O, S, and NH;
wherein Y is selected from: NH2, OH, SH, alkyl, alkenyl, alkynyl, cycloalkyl, cycloalkenyl, cycloalkynyl, and aryl;
wherein W is selected from NH, CH2, S, and O;
wherein X is selected from S, O, CH2, and NH;
wherein n is 0, 1, 2, or 3; and
a pharmaceutically acceptable carrier thereof.

In certain aspects, A is an ester or amide derivate of carboxylic acid, sulfonic acid, or phosphoric acid.

According to certain aspects, disclosed is an anticancer composition comprising a compound having the structure:

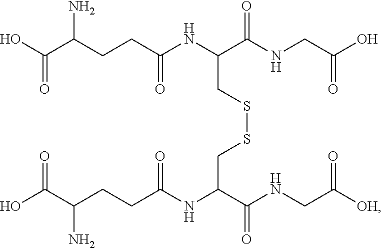 [SMILES: NC(CCC(=O)NC(CSSCC(NC(=O)CCC(N)C(=O)O)C(=O)NCC(=O)O)C(=O)NCC(=O)O)C(=O)O]

and a pharmaceutically acceptable carrier thereof.

According to certain aspects, disclosed is an anticancer composition comprising a compound having the structure:

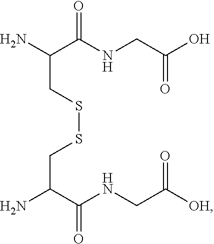 [SMILES: NC(CSSCC(N)C(=O)NCC(=O)O)C(=O)NCC(=O)O]

and a pharmaceutically acceptable carrier thereof.

According to certain aspects, disclosed is an anticancer composition comprising a compound having the structure:

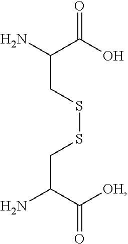 [SMILES: NC(CSSCC(N)C(=O)O)C(=O)O]

and a pharmaceutically acceptable carrier thereof.

According to certain aspects, disclosed is an anticancer composition comprising a compound having the structure:

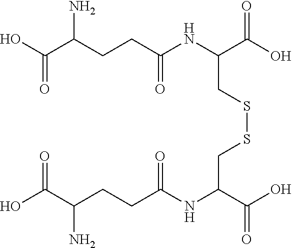 [SMILES: NC(CCC(=O)NC(CSSCC(NC(=O)CCC(N)C(=O)O)C(=O)O)C(=O)O)C(=O)O]

According to certain aspects, disclosed is an anticancer composition comprising a compound having the structure:

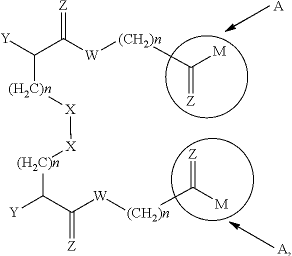

wherein A is a functional group selected from carboxylic acid, sulfonic acid, phosphoric acid, and derivatives thereof;
wherein M is selected from: —OH, —NH2, —SH, —OR, —NHR, —NR1R2, —SR, —R.;
wherein R, R1, and R2 are alkyl, alkenyl, alkynyl, cycloalkyl, cycloalkenyl, cycloalkynyl or aryl;
wherein Z is selected from O, S, and NH;
wherein Y is selected from: NH2, OH, SH, alkyl, alkenyl, alkynyl, cycloalkyl, cycloalkenyl, cycloalkynyl and aryl;
wherein W is selected from NH, CH2, S, and O;
wherein X is selected from S, O, CH2, and NH;
wherein n is 0, 1, 2, or 3; and
a pharmaceutically acceptable carrier thereof.

In certain aspects, A is an ester or amide derivate of carboxylic acid, sulfonic acid, or phosphoric acid.

According to certain aspects, disclosed is an anticancer composition comprising a compound having the structure:

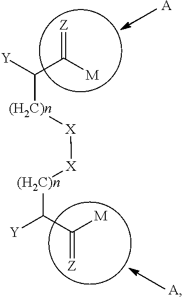

wherein A is a functional group selected from carboxylic acid, sulfonic acid, phosphoric acid, and derivatives thereof;
wherein M is selected from: —OH, —NH2, —SH, —OR, —NHR, —NR1R2, —SR, —R.; wherein R, R1, and R2 are alkyl, alkenyl, alkynyl, cycloalkyl, cycloalkenyl, cycloalkynyl, or aryl;
wherein Z is selected from O, S, and NH;
wherein Y is selected from: NH2, OH, SH, alkyl, alkenyl, alkynyl, cycloalkyl, cycloalkenyl, cycloalkynyl, and aryl;
wherein W is selected from NH, CH2, S, and O;
wherein X is selected from S, O, CH2, and NH;
wherein n is 0, 1, 2, or 3; and
a pharmaceutically acceptable carrier thereof.

In certain aspects, A is an ester or amide derivate of carboxylic acid, sulfonic acid, or phosphoric acid.

According to certain aspects, disclosed is an anticancer composition comprising a compound having the structure:

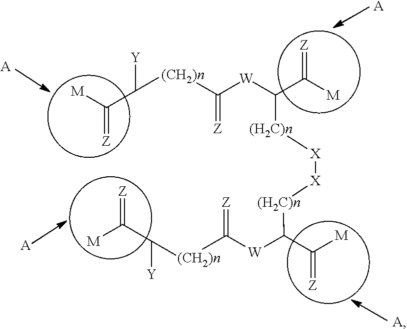

wherein A is a functional group selected from carboxylic acid, sulfonic acid, phosphoric acid, and derivatives thereof;
wherein M is selected from: —OH, —NH2, —SH, —OR, —NHR, —NR1R2, —SR, —R.;
wherein R, R1, and R2 are alkyl, alkenyl, alkynyl, cycloalkyl, cycloalkenyl, cycloalkynyl, or aryl;
wherein Z is selected from O, S, and NH;
wherein Y is selected from: NH2, OH, SH, alkyl, alkenyl, alkynyl, cycloalkyl, cycloalkenyl, cycloalkynyl, and aryl;
wherein W is selected from NH, CH2, S, and O;
wherein X is selected from S, O, CH2, and NH;
wherein n is 0, 1, 2, or 3; and
   a pharmaceutically acceptable carrier thereof.

In certain aspects, A is an ester or amide derivate of carboxylic acid, sulfonic acid, or phosphoric acid.

Also disclosed is a method for treating cancer in a subject in need thereof, the method comprising administering to the subject an effective amount of a composition comprising GSSG, or derivatives thereof, and a pharmaceutically acceptable carrier thereof.

In certain aspects, GSSG derivatives are any GSSG derivatives disclosed herein.

In certain aspects, the composition is administered in a therapeutically effective amount. In further aspects, the composition is administered in a prophylactically effective amount.

In further aspects, the composition administered to the subject may be in a range of about 0.001 mg/kg to about 1000 mg/kg. In further embodiments, the composition is administered at a dose of at least about 1 g/kg per day.

According to certain aspects, administration of the composition to the subject inhibits tumor cell detachment.

In further aspects, administration of the composition to the subject inhibits tumor cell migration. In even further aspects, administration of the composition to the subject inhibits tumor cell invasion. In still further aspects, administration of the composition inhibits tumor growth. In yet further aspects, administration of the composition induces tumor cell apoptosis.

According to certain aspects, the subject has been diagnosed with melanoma, breast cancer, lung carcinoma, pancreatic carcinoma, renal carcinoma, ovarian, prostate or cervical carcinoma, glioblastoma, or colorectal carcinoma, cerebrospinal tumor, head and neck cancer, thymoma, mesothelioma, esophageal cancer, stomach cancer, liver cancer, pancreatic cancer, bile duct cancer, bladder cancer, testicular cancer, germ cell tumor, ovarian cancer, uterine cervical cancer, endometrial cancer, lymphoma, acute leukemia, chronic leukemia, multiple myeloma, sarcoma, or any combination thereof.

In certain aspects, the method further comprises administering the composition as a bolus and/or at regular intervals. In certain aspects, the disclosed method further comprises administering the composition intravenously, intraperitoneally, intramuscularly, orally, subcutaneously, or transdermally.

According to certain embodiments, the disclosed method further comprises administering the composition in conjunction with at least one other treatment or therapy. In certain aspects, the at least one other treatment or therapy comprises co-administering an anti-neoplastic agent. In certain aspects, the other treatment or therapy is chemotherapy.

According to certain further embodiments, the method further comprises diagnosing the subject with cancer. In further aspects, the subject is diagnosed with cancer prior to administration of the composition. According to still further aspects, the method further comprises evaluating the efficacy of the composition. In yet further aspects, evaluating the efficacy of the composition comprises measuring tumor size prior to administering the composition and measuring tumor size after administering the composition. In even further aspects, evaluating the efficacy of the composition occurs at regular intervals. According to certain aspects, the disclosed method further comprises optionally adjusting at least one aspect of method. In yet further aspects, adjusting at least one aspect of method comprises changing the dose of the composition, the frequency of administration of the composition, or the route of administration of the composition.

### Examples

The following examples are put forth so as to provide those of ordinary skill in the art with a complete disclosure and description of certain examples of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

### Preparation of Positively Charged GSSG Liposomes

Positively charged GSSG liposomes were prepared by using a dehydration/rehydration freeze dry method. Twenty mg of dimethyldioctadecylammonium bromide (DDAB), 70 mg of lechithin, and 10 mg of cholesterol was added to 10 mL of chloroform in a 100 mL flask. Solvent was slowly removed through a rotavapor at 45° C. at reduced pressure to complete dryness and the flask is then placed in a vacuum desiccator overnight to remove residual solvent. The lipids in the flask were dispersed in 10 mL of phosphate buffered saline (PBS) containing GSSG (100 mg/mL). This dispersion was sonicated for 20 min. The sonicated dispersion was freeze-dried to result in dried crude GSSG liposomes and stored at −80° C. These crude GSSG liposomes were reconstituted with 10 mL of water and passed a sepharose column (PD-10 Columns, Sephadex G-25M, GE Healthcare Biosciences, Pittsburgh, Pa.) to remove un-capsulated GSSG. The particle size and zeta potential of GSSG liposomes were determined to be 190 nm and ˜+70 mV respectively using a zeta potential instrument (Malvern Zetasizer Nano-ZS, Malvern Instruments, Worcestershire, United Kingdom).

GSSG Liposomes Deliver GSSG into Cells

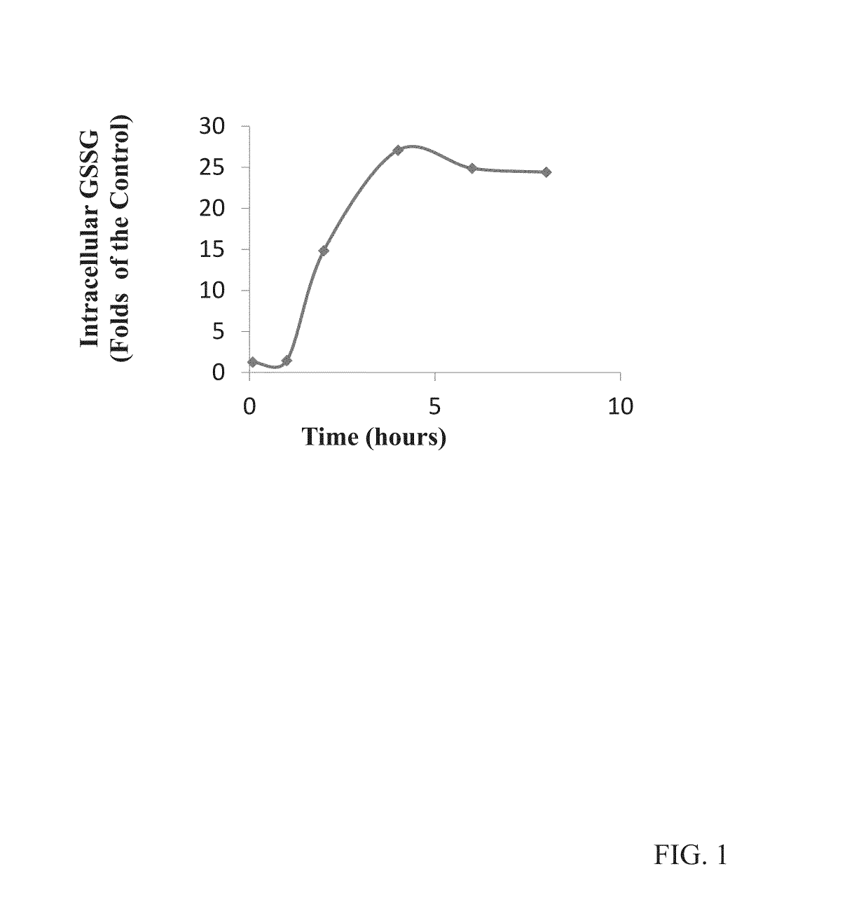

When NCI-H226 cells (4×10⁶) were treated with GSSG liposomes in RPMI 1640 medium (the final GSSG concentration was 1 mg/mL in the medium) at 37° C. in a 75 cm² flask in a CO₂ incubator, intracellular GSSG was increased over time and reached to 25-fold of the control at 4 h (FIG. 1). Intracellular GSSG was determined by LC/MS. No increase in intracellular GSSG was observed when aqueous GSSG solution (1 mg/mL) was incubated with cells confirming that GSSG itself could not penetrate cell membrane while GSSG liposomes effectively delivered GSSG into cells. As best shown in FIG. 1, GSSG liposomes successfully deliver GSSG into cells.

GSSG Liposomes Completely Prevented Cells from Detachments

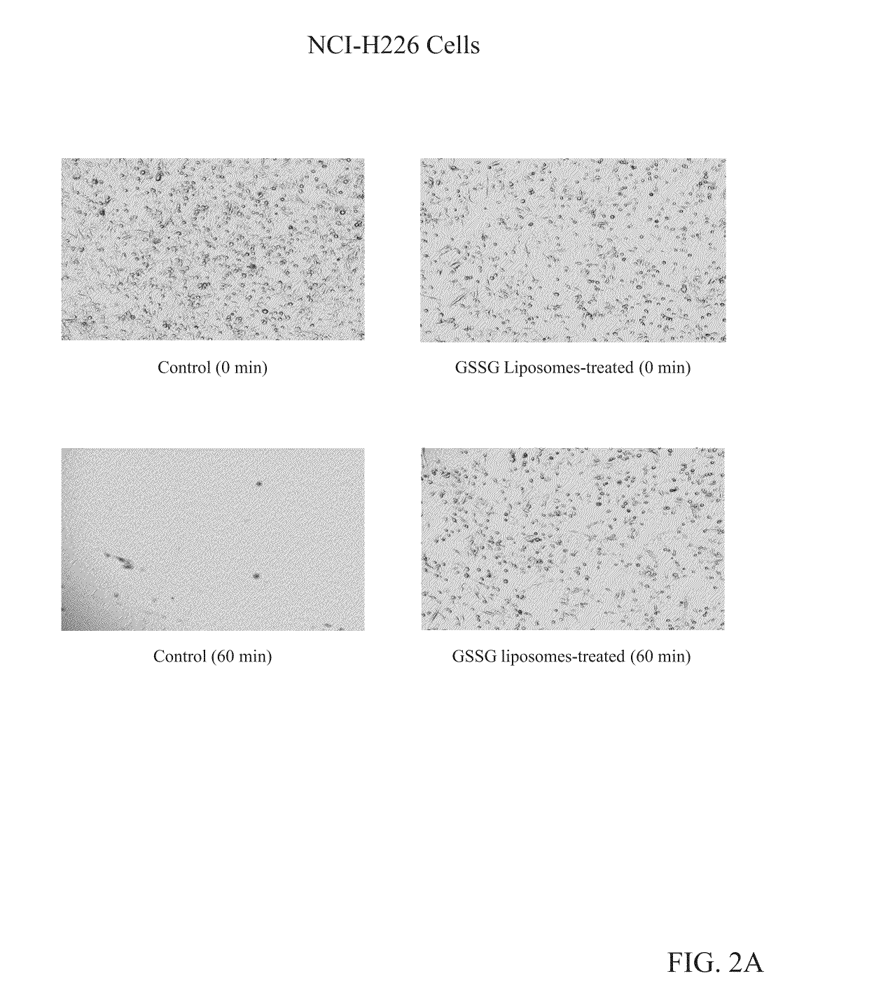

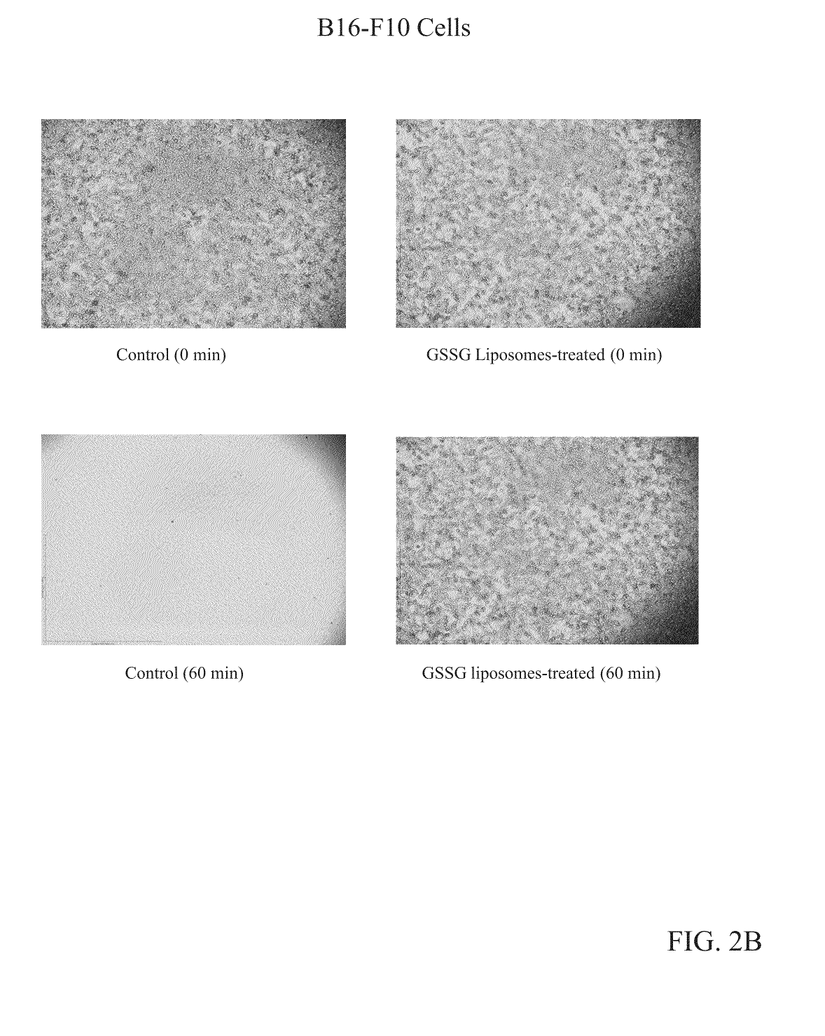

The effect of GSSG liposomes on cell detachment was studied through a controlled trypsinization experiment. Cells were treated with blank liposomes (control) or 1 mg/mL GSSG liposomes (treatment) for 24 h. The cells were subjected to controlled trypsinization to observe for detachment. As shown in FIGS. 2A and 2B when cells were treated with GSSG liposomes, the cells were found to be resistant toward trypsin-mediated detachment while the cells in the control detached readily [two cancer cell lines were tested: NCI-H226 human lung cancer (FIG. 2A) and B16-F10 murine melanoma cell line (FIG. 2B)]. This was further confirmed by the observation that no cells were found in the supernatant of the GSSG liposome-treated samples, while attached cells were detached into the supernatant of the control samples over the time (Table 1).

TABLE 1A
Number of NCI-H226 cells found in the supernatant obtained from
centrifugation of the culture medium after trypsinization.
Number of cells in supernatant

| Time | 10 min | 30 min | 60 min |
|---|---|---|---|
| control | 185000 | 320000 | 295000 |
| GSSG liposomes-treated | Not detected | Not detected | Not detected |

TABLE 1B
Number of B16-F10 cells found in the supernatant obtained from
centrifugation of the culture medium after trypsinization.
Number of cells in supernatant

| Time | 10 min | 30 min |
|---|---|---|
| control | 185000 | 349000 |
| GSSG liposomes-treated | Not detected | Not detected |

### GSSG Liposomes Completely Blocked Cell Migration

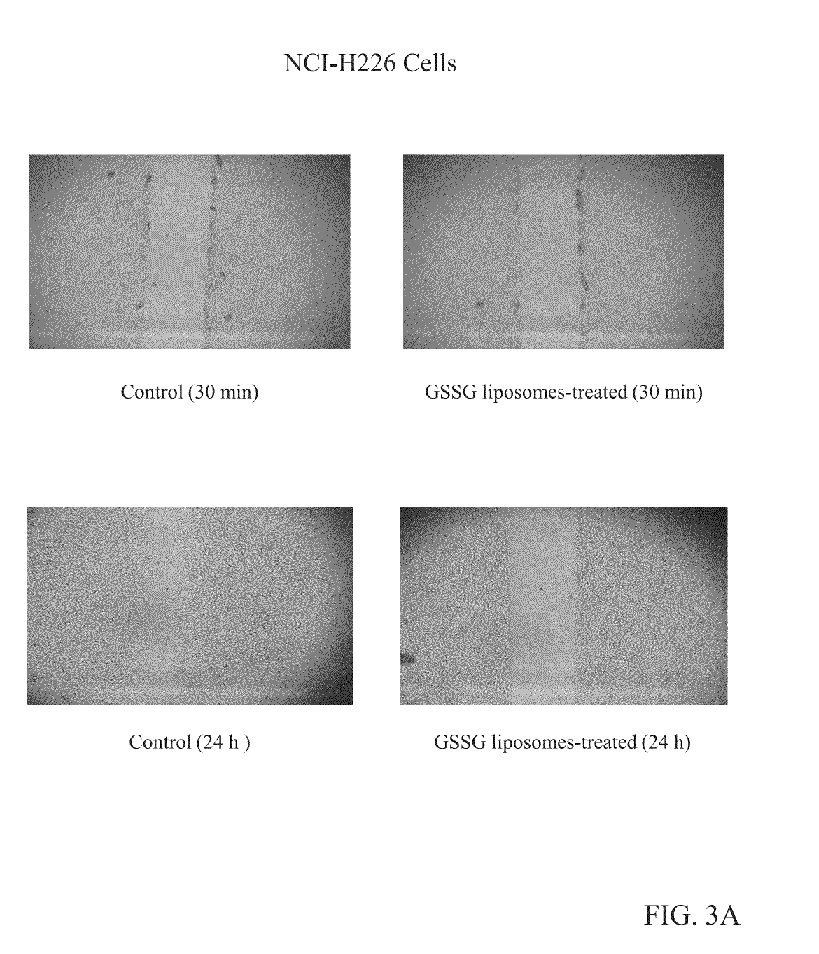

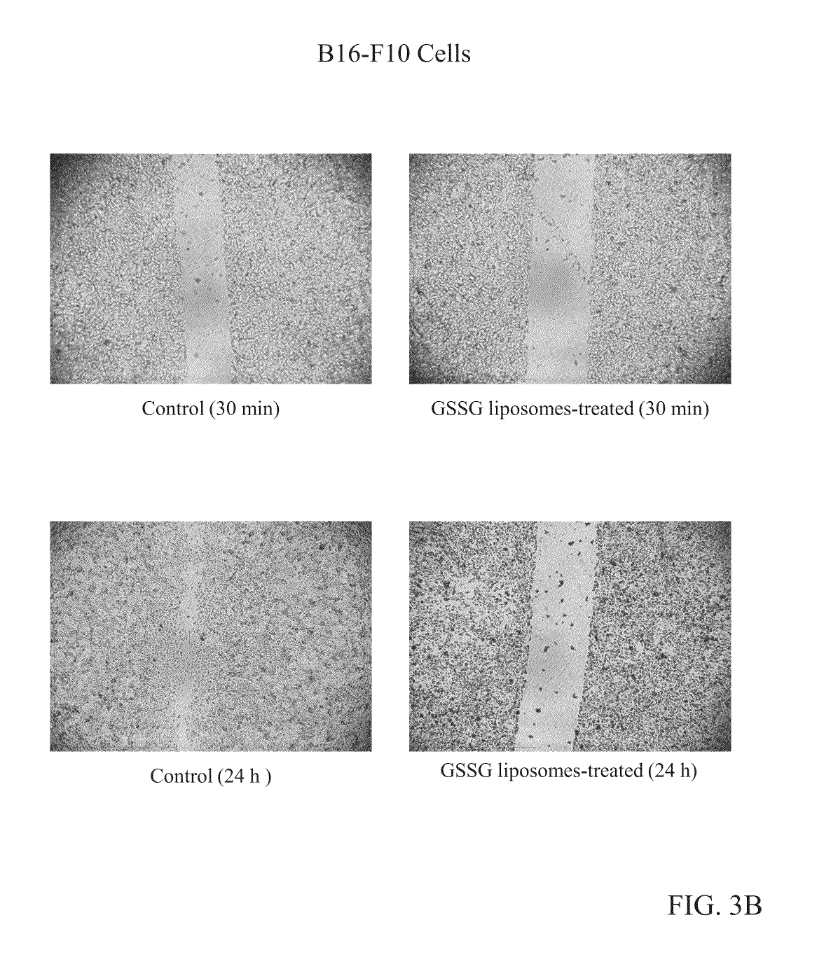

The effect on cell migration was investigated through a “wound healing” assay in which a “wound” was created in the attached cells before a treatment started. As shown in FIGS. 3A and 3B, GSSG liposomes prevented the cells from filling (migrating into) the wound. Cells were seeded into each well of a 12-well plate in RPMI 1640 medium supplemented with 10% FBS for 24 h for attachment. A “wound” was created by scraping the confluent portion of cells with the tip of a sterile 200 μl plastic pipette tip. The residual monolayers were washed twice and overlaid with fresh growth media containing GSSG liposomes (1 mg/mL GSSG) for treatment or blank liposomes for control. The wound gap was photographed under a phase contrast microscope. Data presented were from one of the three representative experiments. GSSG liposomes completely blocked cells from migration in both NCI-H226 cells (FIG. 3A) and B16-F10 cells (FIG. 3B).

### In Vitro Cancer Growth Inhibition and Apoptosis

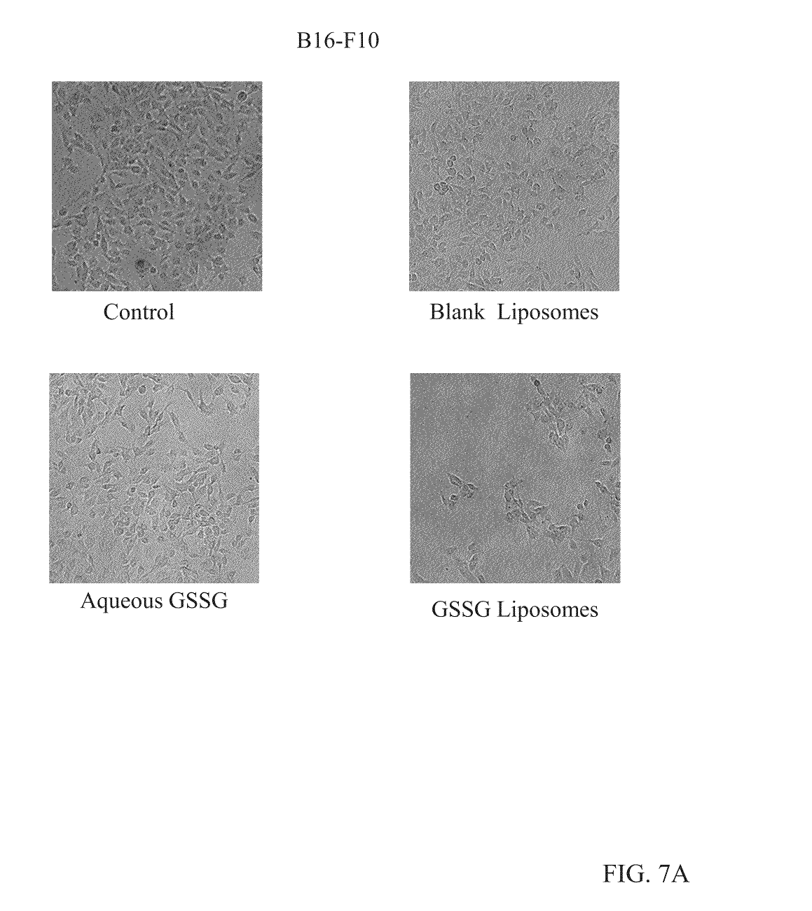

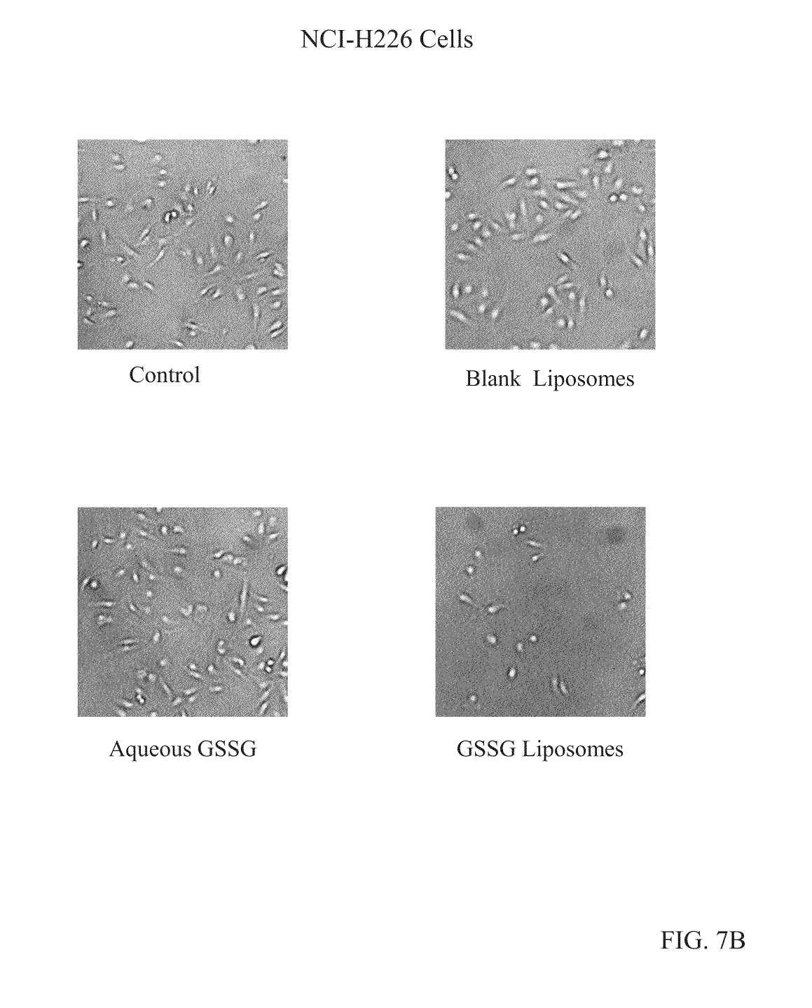

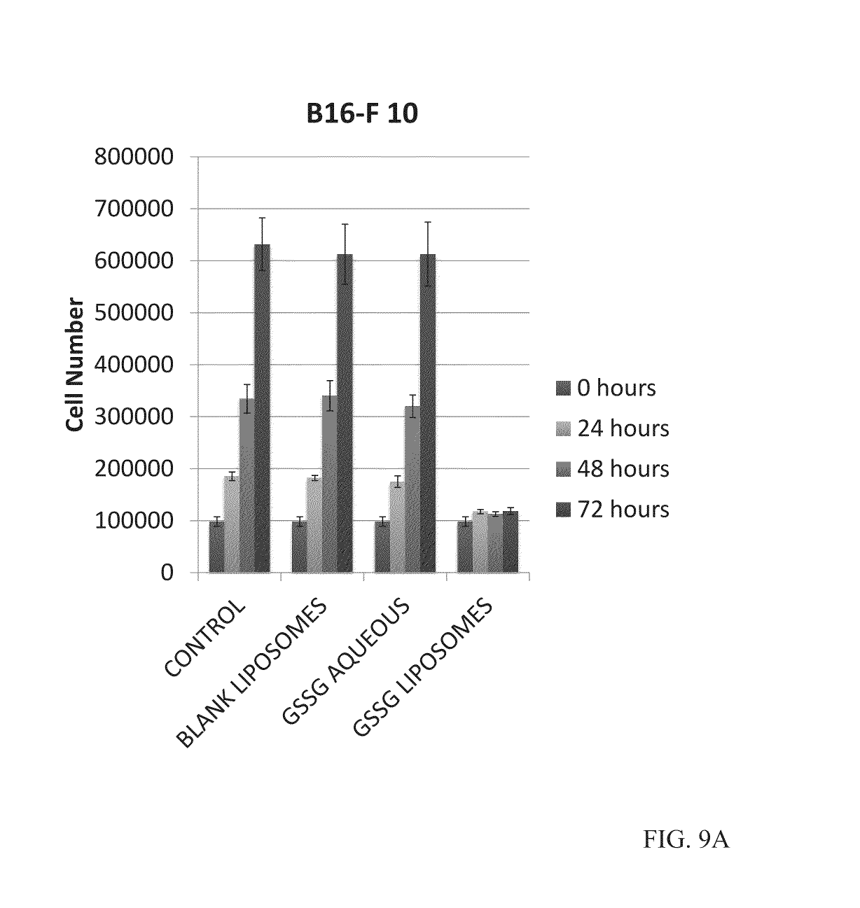

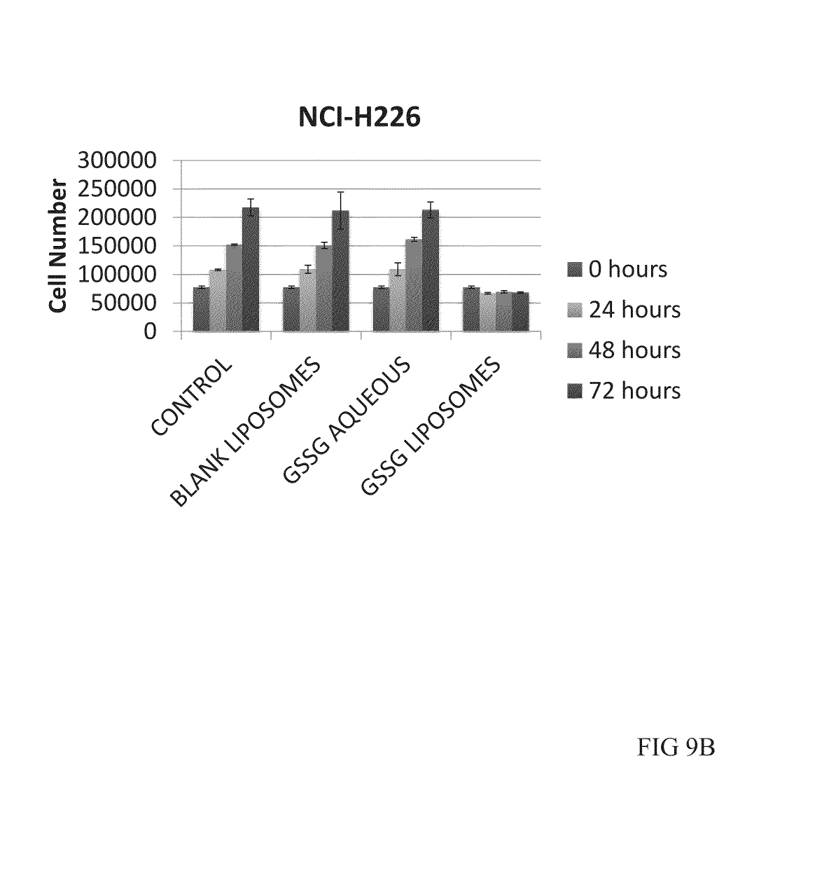

When B16-F10 cells were treated with GSSG liposomes for 24 h, cells remained attached and also appeared to stop growing. As shown in FIG. 7A, a Trypan blue assay revealed that the cells were >95% viable although morphological changes were observed. To investigate whether the effect was limited to B16-F10 cells, the same experiment was conducted with NCI-H226 cells. As shown in FIG. 7B, similar phenomena were observed with NCI-H226 cells. The visual observation of a halt in cell growth and a >95% cell viability by the Trypan blue assay led us suspect that GSSG liposomes might exhibit a cytostatic effect. To determine whether GSSG liposomes exhibit a cytostatic effect, cells, after being treated with GSSG liposomes for 0 h, 24 h, 48 h, and 72 h, were collected with a sterile cell scraper and counted using a cell counter. As shown in FIG. 9, GSSG liposomes stopped the growth of both B16-F10 (FIG. 9A) and NCI-H226 (FIG. 9B) cells when compared with the control in which cells were treated with the growth medium only (control-1). In the meantime, cells treated with blank liposomes (control-2) and aqueous GSSG (control-3) exhibited the same growth rates as those in control-1 confirming that it was GSSG being delivered into cells that produced the cell growth halt effect (FIG. 9). Interestingly unlikely at 24 h, the Trypan blue assay revealed that >95% cells were dead for cells treated with GSSG liposomes for 48 and 72 h while cells in all controls remained >95% alive. These results suggested that GSSG liposomes stopped cell growth completely in the first 24 h and eventually led to cell death. The data also raised a question whether the cells could resume growth if the GSSG liposomes treatment medium was removed after 24 h treatment. An experiment was conducted in which cells (B16-F10 and NCI-H226) were first treated with GSSG liposomes for 24 h, followed by replacement of the GSSG liposomes treatment medium with fresh growth medium, and then allowed to grow for an additional 24 h. To our surprise, no cell growth was observed and, rather, >95% cells were found to be dead by the Trypan blue assay at the end of the experiment. In the meantime, cells in controls 1-3 [growth medium containing PBS (control-1), growth medium containing blank liposomes (control-2), and growth medium containing aqueous GSSG (control-3)] grew normally with >95% cell viability. This experiment indicates that after 24 h treatment with GSSG liposomes, cells were probably on an irreversible path to death despite the fact that they were >95% alive by the Trypan blue assay.

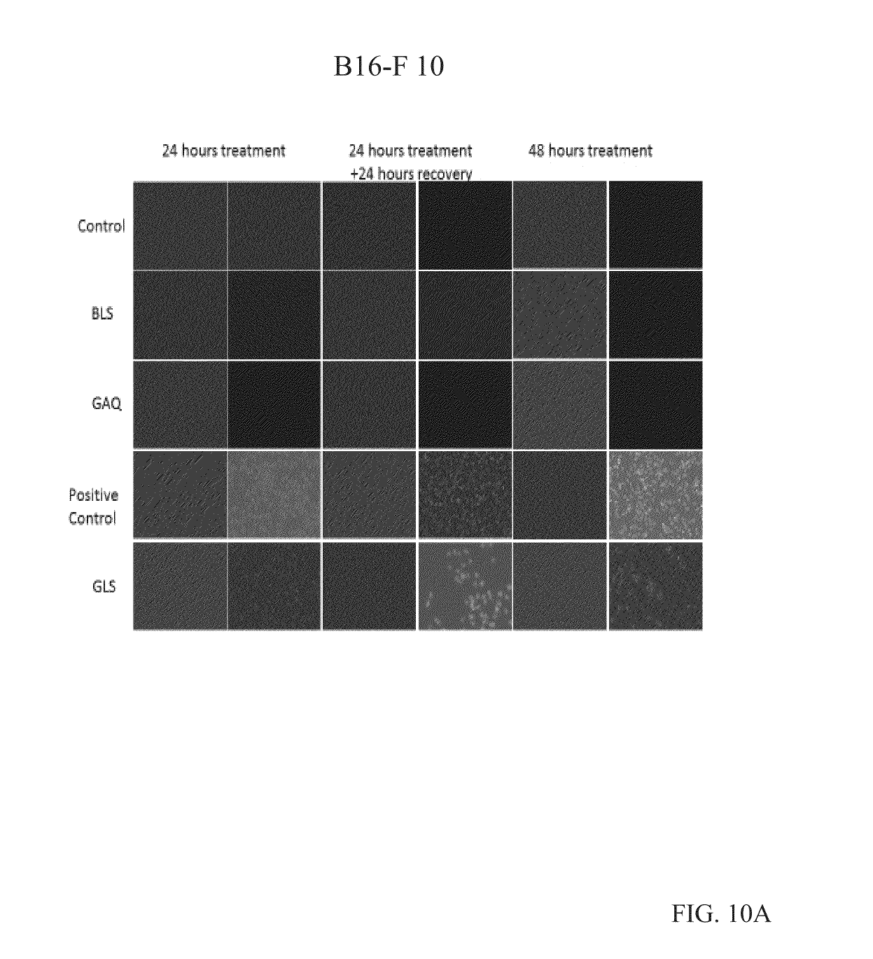

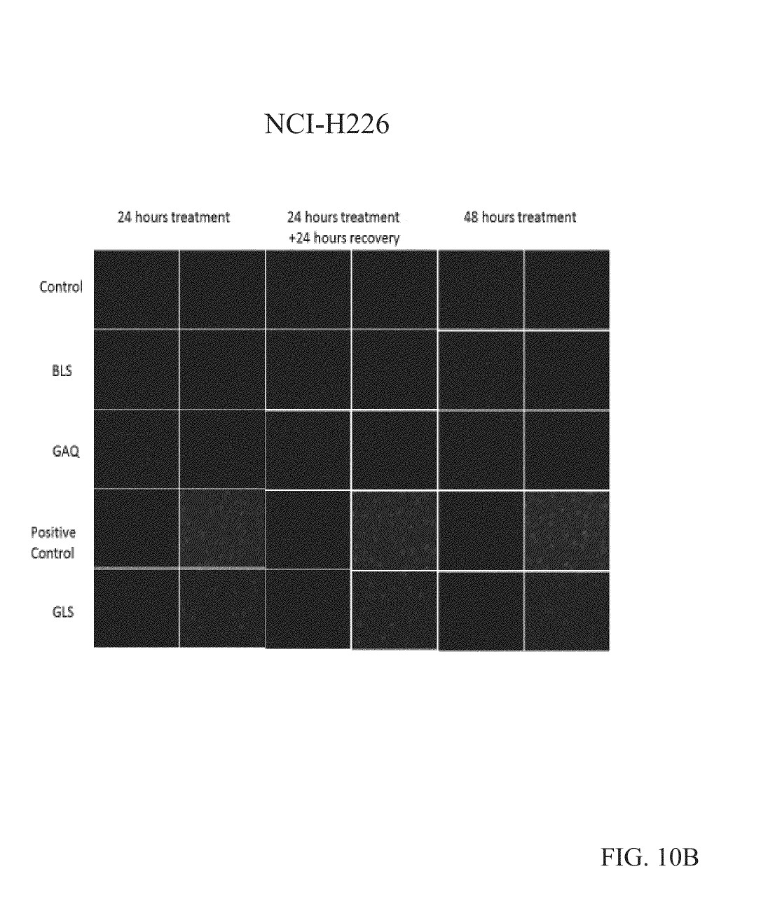

To further investigate the status of cells treated with GSSG liposomes for 24 h and 48 h, the TUNEL assay was employed. The TUNEL assay would reveal the apoptosis status of cells. FIGS. 10A and 10B show the effects of various treatments on apoptosis of cancer cells (FIG. 10A: B16-F10; FIG. 10B: NCI-H226). Cells were treated with a treatment [control-1 (CTRL): medium containing PBS; Control-2 (BLS): blank liposomes; control-3 (GAQ): aqueous GSSG (1 mg/mL); positive control: TACS-Nuclease™ Buffer; or GSSG liposomes (GLS) (1 mg/mL)] in a 24-well plate in a CO₂ incubator at 37° C. as described in FIG. 9 except the TUNEL assay was conducted instead of the Trypan blue assay. Images were obtained on an inverted fluorescence microscope (Ziess, Observer.A1, AX-10) connected to a camera (Ziess, Axiocam MRc5). The data demonstrate that no cell apoptosis was observed for cells in controls 1-3. As expected, greater than 95% cells were found to undergo apoptosis when treated with TACS-Nuclease™ Buffer. Similar to the positive control, greater than 95% of cells were found to undergo apoptosis for cells treated with GSSG liposomes at both 24 h and 48 h. This is interesting since cell viability, determined by the Trypan blue assay, was found to be >95% at 24 h for cells treated with GSSG liposomes.

The discrepancy in percentage of cell viability determined by the Trypan blue and the percentage of cells undergoing apoptosis at 24 h is likely related to the assay mechanisms. The Trypan blue assay determines the cell viability based on cell membrane integrity. The TUNEL assay determines cell apoptosis based on formation of DNA fragments. It is likely that the cell membrane remained intact for cells treated with GSSG liposomes for 24 h even though the cells were undergoing apoptosis already. Therefore, it is concluded that the observation of no cell number increase for cells treated with GSSG liposomes was due to the reason that the cells were undergoing apoptosis not due to the reason that cells were halting their growth.

### Effects on Cell Cycle Distribution

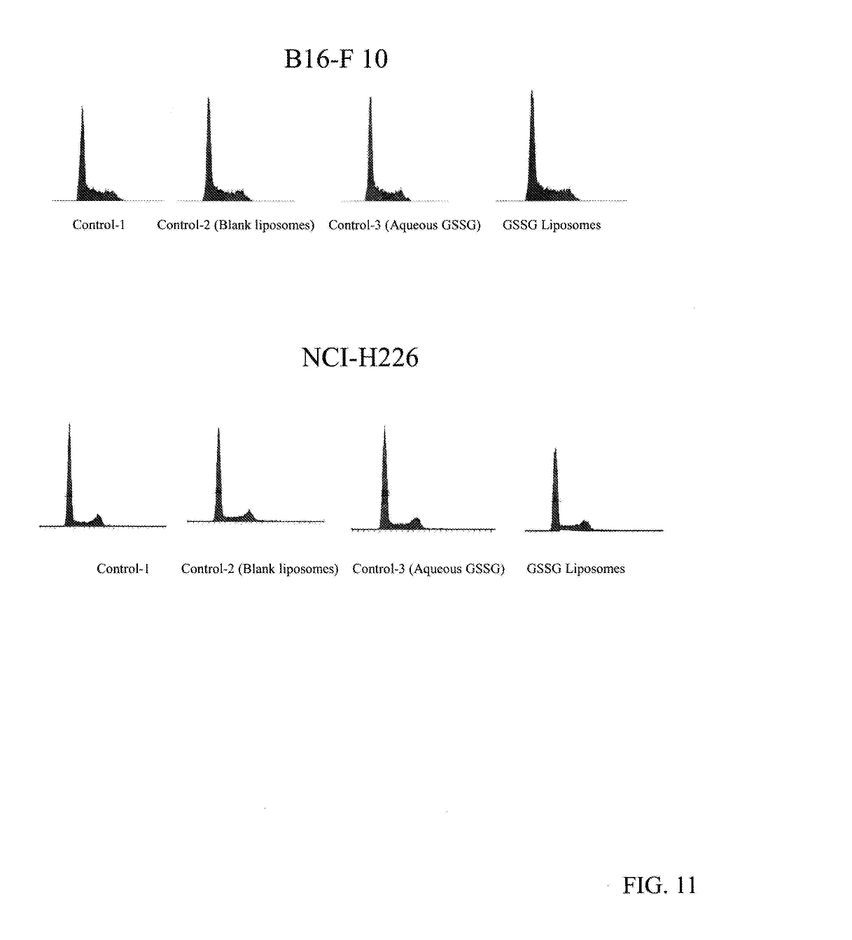

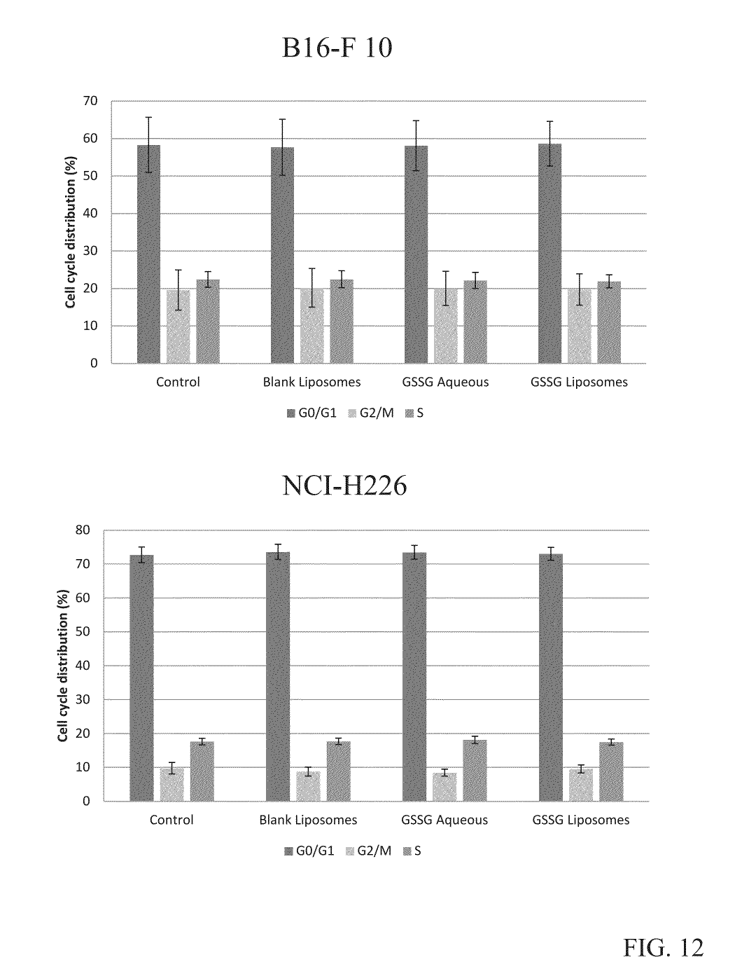

The effect of GSSG liposomes on cell cycle distribution was investigated. When cells were treated with GSSG liposomes (1 mg/mL) for 24 h and 48 h, both NCI-H226 and B16-F10 cells showed no change in cell cycle. No effects were observed with blank liposomes or GSSG aqueous solution (1 mg/mL) either. FIG. 11 provides histograms of B16-F10 and NCI-H226 cells from one representative experiment of a triplet in which cells were treated with various treatments for 24 h. The percentages of cells in different cell cycle are presented in FIG. 12.

### Cancer Metastasis Inhibition by GSSG Liposomes

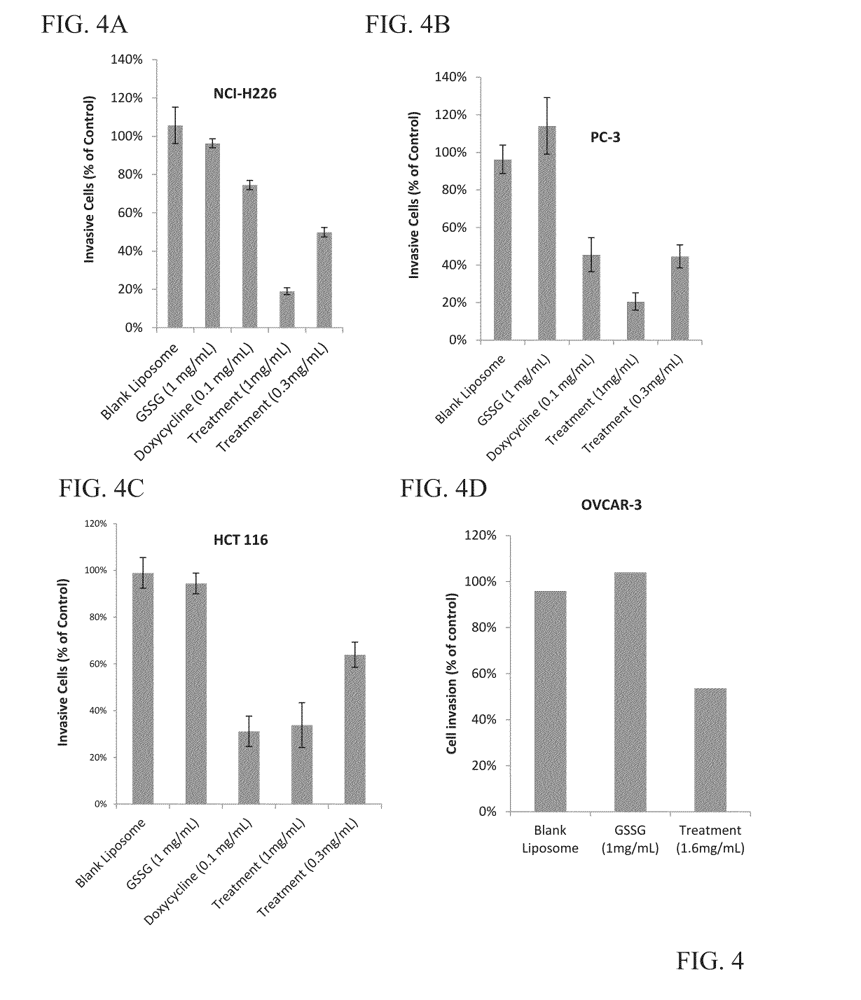

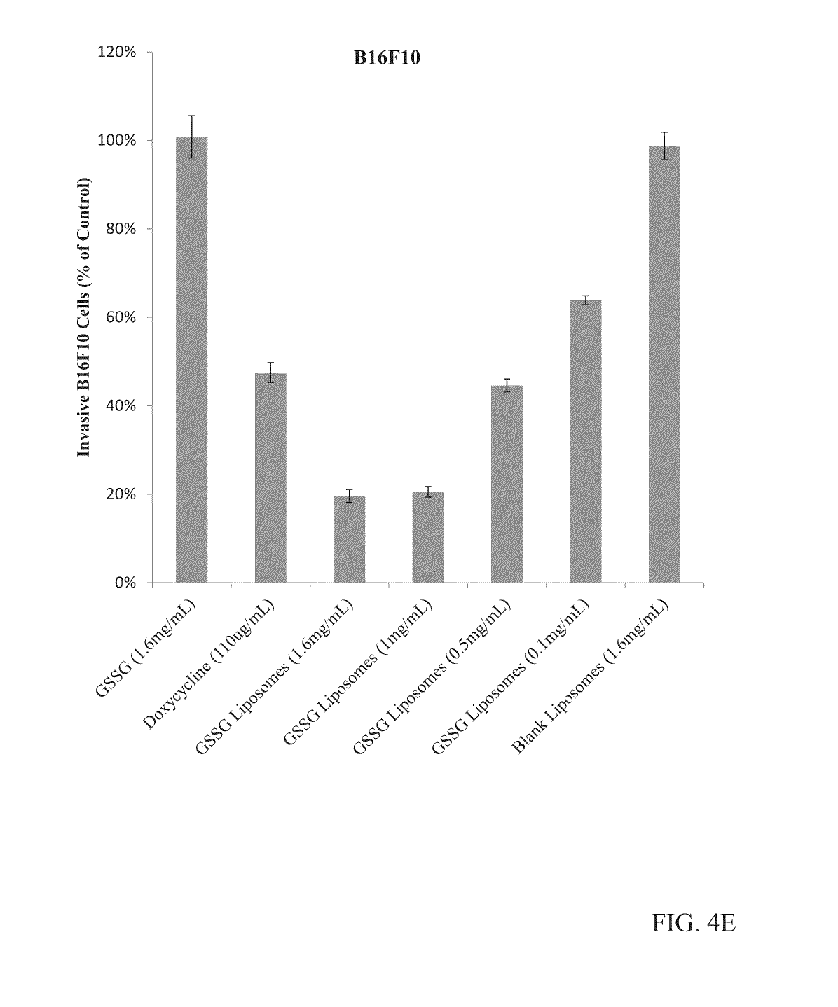

Five cancer cell lines were tested: lung cancer NCI-H226, prostate cancer PC-3, ovarian cancer OVCAR-3, colon cancer HCT 116, and B16-F10 murine melanoma cell line. The impact of GSSG liposomes on the invasive ability was studied using a commercially available cell invasion kit [Matrigel Invasion Chambers was used (8 μm pore; BD Biosciences)]. In a cell invasion assay set consisting of a cell culture chamber, an invasion chamber, and an assay chamber, cells (50,000) were incubated in the culture chamber for 24 h at 37° C. in a CO₂ incubator. At the end of incubation, cells reaching to the assay chamber through the invasion chamber were quantified through a fluorescence microplate reader by using Calcein-AM as a cell number detecting agent. GSSG liposomes effectively inhibited the invasion property of the tested cancer cells. In the study, doxycycline, a known cancer invasion inhibitor, was used as a positive control, aqueous GSSG solution and blank liposomes were used as controls. As shown in FIG. 4, GSSG liposomes inhibited the invasion of the cancer cells more effectively than doxycycline while GSSG alone or blank liposomes exhibited no inhibitory effects.

GSSG Liposomes Completely Prevented Cancer Cells from Metastasis in Mice

Mice were housed four in a cage and received food and water ad libitum. All mice were used at approximately 7-10 weeks of age and given at least one week break after arrival. All experimental protocols were approved by the Institutional Animal Care and Use Committee. Procedures for murine lung metastasis assays reported previously were followed with minor modification [Bezault, J., Bhimani, R., Wiprovnick, J., and Furmanski, P. (1994) Cancer Res 54, 2310-2312; Kalland, T. (1986) Cancer Res 46, 3018-3022; Schultz, R. M., Silberman, S., Persky, B., Bajkowski, A. S., and Carmichael, D. F. (1988) Cancer Res 48, 5539-5545]. Mice were divided into 7 mice per group for treatment. B16-F10 cells were grown and maintained as monolayers in RPMI 1640 medium supplemented with 10% FBS and 1% penicillin/streptomycin in 5% CO₂ at 37° C. The cells were then subjected to FBS starvation in RPMI 1640 medium without FBS for 24 h. Cells were harvested through trypsinization and adjusted to a density of 950,00 cells/mL with serum free medium. Cells were pretreated with different treatment as indicated in Table 1 prior to be injected to mice. Each mouse received a 0.2 mL aliquot of cells (175,000/mouse) through a tail vein. Treatments, as indicated in Table 2, started 24 h after introduction of the cells, and continued daily for 5 days. The weight of mice was recorded daily. Mice were euthanized by cervical dislocation under isoflurane on day 21, and the lungs were removed, washed in PBS, and fixed with buffered formalin solution (Fisher Scientific) for 24 h before being photographed. Tumor nodules on lung surface were counted under a magnifier.

TABLE 2
Mouse treatment protocol

| | | Control-2 | Control-3 | Treatment-1 | | Positive |
|---|---|---|---|---|---|---|
| Groups | Control-1 | (Pretreatment) | (Pretreatment) | (Pretreatment) | Treatmet-2 | control |
| B16-F10 | PBS | Blank | Aqueous GSSG | GSSG | PBS | PBS |
| cells | | liposomes | (1 mg/mL) | liposomes | | |
| pretreated | | | | (1 mg/mL) | | |
| for 24 h | | | | | | |

B16-F10 cells injected through a tail vein

| | | | | | | |
|---|---|---|---|---|---|---|
| Treatment | PBS by | Blank | Aqueous GSSG | GSSG | GSSG | Dacarbazine |
| daily for 5 | i.v. | liposomes by | by i.v. | liposomes | liposomes | by i.p. |
| days | | i.v. | (1 g/kg) | by i.v. | by i.v. | (50 mg/kg) |
| | | | | (1 g/kg) | (1 g/kg) | |

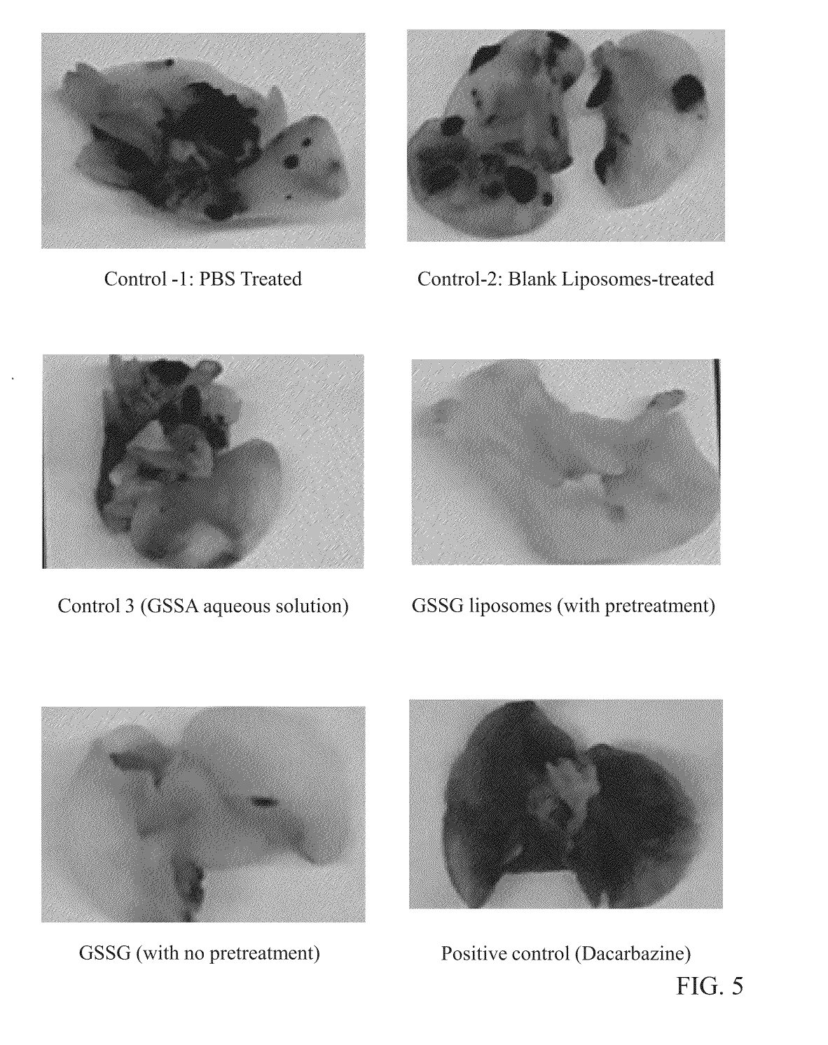

FIG. 5 presents the representative photographs of the lungs dissected from mice with different treatments. As shown in the figure, no lung metastasis was observed in mice treated with GSSG liposomes (in both pretreatment and without pretreatment groups) while significant lung metastasis was observed in control 1 (PBS) and control 2 (blank liposome treated). The rationale to pretreat the cancer cells before being injected to mice was based on the in vitro results that cells lost the abilities to detach and migrate after 24 h treatment. We would like to see whether a pretreatment would be required for the in vivo anti-metastatic effect. Our data did not show a significant difference in the anti-metastatic effects between the pretreatment group and no pretreatment group. No lung metastasis was visually observed with the positive control (Dacarbazine). Visual observation also revealed that much less lung metastasis occurred with the mice treated with aqueous GSSG (control 3) when compared with control 1 and control 2, which was not expected since aqueous GSSG did not produce any effect on cell detachment, migration and invasion in the in vitro experiments. The effect was further confirmed by the metastatic tumors counted under a magnifier (Table 3). About 60% metastasis inhibition was produced by aqueous GSSG. As shown in Table 3, blank liposomes showed no effect on lung metastasis. The GSSG pretreatment group was observed to be completely free of lung metastasis. Metastatic tumors were spotted in one mouse in the GSSG liposomes without pretreatment group and in one mouse in the Dacarbazine group.

TABLE 3
Number of metastatic tumors in murine lungs

| Mouse | Average |
|---|---|
| PBS (control 1) | 52.7 ± 10.1 |
| BLS (control 2) | 40.9 ± 17.7 |
| GSSG aqueous solution | 14.1 ± 8.3 |
| (control 3) | |
| GSSG liposomes (1 g/kg, | 1 ± 2.6 |
| no pretreatment) | |
| GSSG liposomes (1 g/kg, | 0 ± 0 |
| with pretreatment) | |
| Dacarbazine (50 mg/kg) | 0.8 ± 2.0 |

### GSSG Liposomes Significantly Inhibited the Growth of Cancer Cells in Mice

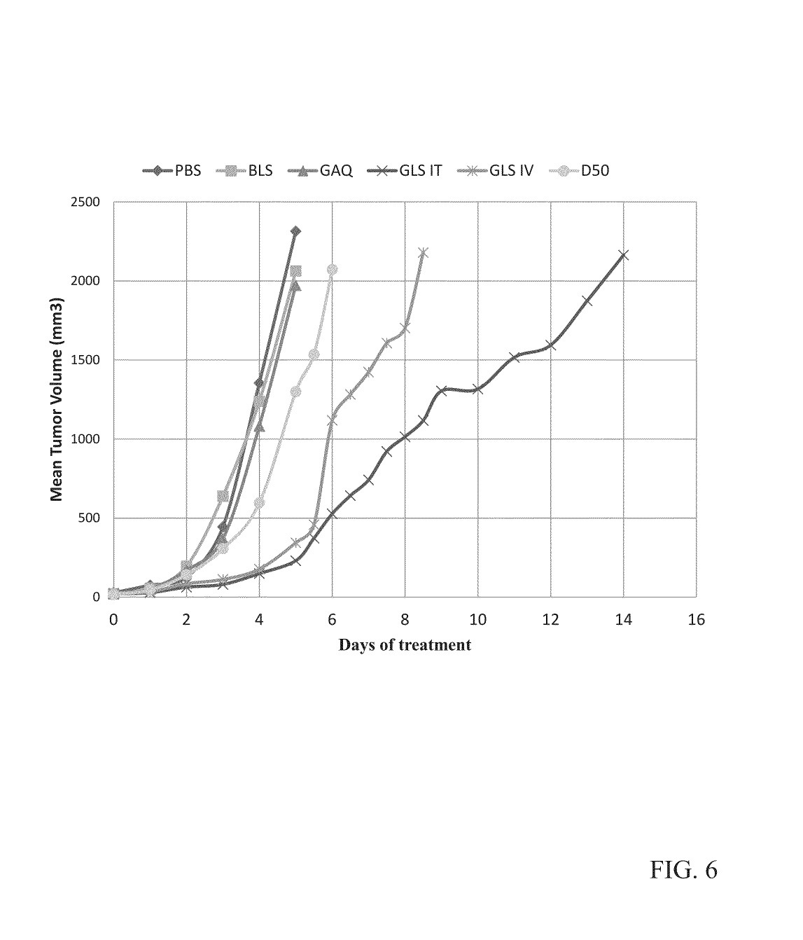

The in vivo effect of GSSG liposomes on tumor growth was investigated with a murine melanoma model with female C57BL/6 mice (6 mice per treatment group) employed by Wack and colleagues (Wack et. al., (2001) Melanoma research 11, 247-253). The hair of the right flank of a mouse was removed by use of Nair™ hair removing cream one day before inoculation of B16-F10 cells (2 million in 50 μL PBS) through subcutaneous injection. A treatment started daily after the tumors had reached an average volume of 25 mm³. The treatment continued for 5 days followed by a two-day break and an additional three-day treatment. Mice were weighed daily and the tumor size was measured daily. The tumor volume was calculated based on the formula of 0.5×L×W². Mice were sacrificed when the tumor volume reached an average volume of 2,000 mm³. As shown in FIG. 6, tumors grew rapidly in mice treated with PBS (control-1), blank liposomes (BLS) (control-2), or aqueous GSSG (GAQ) (1 g/kg) (control-3), and reached the volume of 2,000 mm³ in 5 days. No significant difference was observed for tumor growth rates with these three controls. Dacarbazine (50 mg/kg) was employed as a positive control. Dacarbazine delayed tumor growth slightly (FIG. 6). Dacarbazine is one of the therapeutic agents used for the treatment of melanoma and was investigated for its effect on the growth of subcutaneously implanted melanoma by Wack (Wack et. al., (2001) Melanoma research 11, 247-253). In contrast, GSSG liposomes administered by either IV or intratumoral injection significantly delayed tumor growth with intratumoral injection to be more effective. The average time required for tumors to reach the volume of 2,000 mm³ was ˜8.5 days (by IV) and 14 days (by intratumoral injection) respectively for mice treated GSSG liposomes (1 g/kg) vs 5 day for the control. The body weights of mice were recorded and no body weight difference was observed when compare mice treated with GSSG liposomes vs those in control groups.

### In Vivo Toxicity Study

An in vivo toxicity study was conducted by treating CD-1 female mice with GSSG liposomes (0.6 g/kg, 1.2 g/kg, 3 g/kg, and 6 g/kg) daily for continuous 5 days followed by a two-day break for a total of 12 days. Mice were closely monitored for any abnormal behavior especially in the first two hours of GSSG liposomes administration. The weight of mice was recorded daily. On day 5, one mouse from each group was subjected to pathologically examination. At the end of the experiment (day 12), all mice were subjected for pathological examination. No sign of abnormal behavior was observed for all mice treated with GSSG liposomes. No weight difference between the treated and control was observed either. Pathological examination of liver, heart, kidney, brain, lung, intestine, and stomach from mice treated with GSSG liposomes revealed that all these organs were normal at the dosages employed.

### GSSG Liposomes Inhibit Microtubule Polymerization

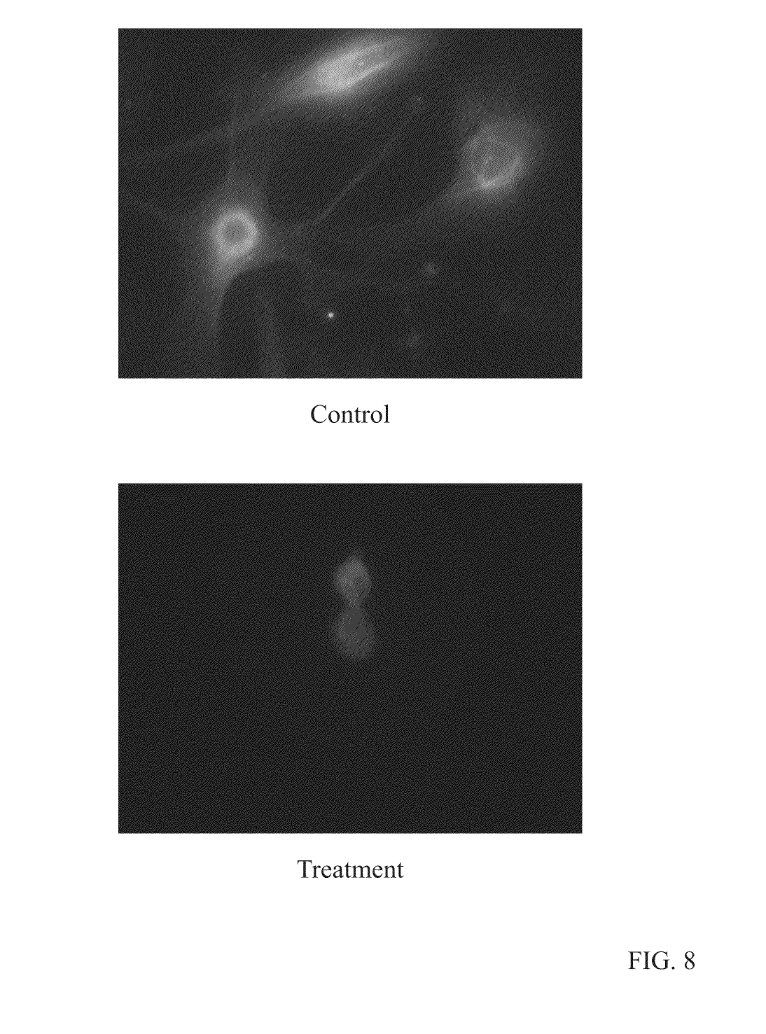

FIG. 8 shows the effect of GSSG liposomes on microtubule polymerization. NCI-H226 cells (2500 cells) were incubated in the presence (treatment) or absence (control) of GSSG liposomes (the final GSSG concentration in the medium is 1 mg/mL) at 37° C. in a CO₂ incubator for 3 h. The cells were fixed with 4% paraformaldehyde, permeabilized with a cell permeable solution (0.1% Na-citrate, 0.1% Triton-X-100 in 1×PBS), and treated with mouse monoclonal anti-a-tubulin-FITC and DAPI. The images were obtained through a Zeiss AX-10 microscope (Carl Zeiss, Inc., Jena, Germany). When NCI-H226 cells were incubated in the presence of GSSG liposomes in a CO₂ incubator at 37° C. for 3 h, microtubules were found depolymerized indicating an antimitotic effect of GSSG liposomes.

Although the present invention has been described with reference to preferred embodiments, persons skilled in the art will recognize that changes may be made in form and detail without departing from the spirit and scope of the invention.

What is claimed is:

1. An anticancer composition comprising glutathione disulfide (GSSG) and a pharmaceutically acceptable carrier thereof.

2. The composition of claim 1, wherein the carrier is a liposome.

3. The composition of claim 2, wherein the liposome is a positively charged liposome.

4. The composition of claim 1 wherein the carrier is a micelle.

5. An anticancer composition comprising a compound having the structure: 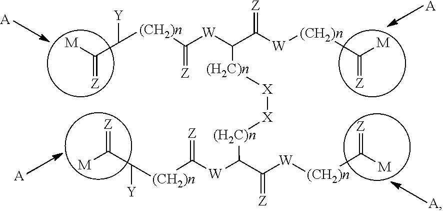
wherein A is a functional group selected from carboxylic acid, sulfonic acid, phosphoric acid, and derivatives thereof;
wherein M is selected from: —OH, —NH2, —SH, —OR, —NHR, —NR1R2, —SR, —R;
wherein R, R1, and R2 are alkyl, alkenyl, alkynyl, cycloalkyl, cycloalkenyl, cycloalkynyl or aryl;
wherein Z is selected from O, S, and NH;
wherein Y is selected from: NH2, OH, SH, alkyl, alkenyl, alkynyl, cycloalkyl, cycloalkenyl, cycloalkynyl, and aryl;
wherein W is selected from NH, CH2, S, and O;
wherein X is selected from S, O, CH2, and NH;
wherein n is 0, 1, 2, or 3; and
a pharmaceutically acceptable carrier thereof.

6. The composition of claim 5, wherein the carrier is a liposome.

7. The composition of claim 6, wherein the liposome is a positively charged liposome.

8. The composition of claim 5 wherein A is an ester or amide derivate of carboxylic acid, sulfonic acid, or phosphoric acid.

9. A method for treating cancer in a subject in need thereof, the method comprising administering to the subject an effective amount of a composition comprising GSSG and a pharmaceutically acceptable carrier thereof.

10. The method of claim 9 wherein the composition is administered in a therapeutically effective amount.

11. The method of claim 10, wherein the composition is administered to the subject at a dose of between about 0.001 mg/kg to about 1000 mg/kg.

12. The method of claim 9, wherein administration of the composition to the subject inhibits tumor cell detachment.

13. The method of claim 9, wherein administration of the composition to the subject inhibits tumor cell migration.

14. The method of claim 9, wherein administration of the composition to the subject inhibits tumor cell invasion.

15. The method of claim 9, wherein administration of the composition inhibits tumor growth.

16. The method of claim 9, wherein administration of the composition induces tumor cell apoptosis.

17. The method of claim 9, further comprising administering the composition intravenously, intraperitoneally, intramuscularly, orally, subcutaneously, or transdermally.

18. The method of claim 9, further comprising administering the composition in conjunction with at least one other treatment or therapy.

19. The method of claim 18, wherein the other treatment or therapy comprises co-administering an anti-neoplastic agent.

20. The method of claim 18, wherein the other treatment or therapy is chemotherapy.
